(19) United States
(12) Patent Application Publication
(10) Pub. No.: US 2025/0017781 A1
(43) Pub. Date: Jan. 16, 2025

(54) METHOD FOR PROVIDING CONTROL DATA FOR AN OPHTHALMOLOGICAL LASER OF A TREATMENT APPARATUS

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/762,050

(22) Filed: Jul. 2, 2024

(30) Foreign Application Priority Data
Jul. 11, 2023 (DE) 10 2023 118 317.6

(51) Int. Cl. A61F 9/008; G16H 20/40; G16H 40/63

(52) U.S. Cl. CPC A61F 9/00827; A61F 9/00804; G16H 20/40; G16H 40/63; A61F 2009/00872; A61F 2009/00878

(57) ABSTRACT

Method for providing control data for an ophthalmological laser (12) of a treatment apparatus (10) for treating a cornea (16) of a human and/or animal eye. As steps, the method performed by a control device (18) comprises determining (S10) eye parameters from predetermined examination data; determining (S12) initial correction parameters for treating the cornea (16) depending on the eye parameters; determining (S14) treatment result data by a database (24), which includes predetermined treatment results of preceding corneal treatments, wherein the treatment result data is retrieved from treatments with comparable eye parameters and initial correction parameters; determining (S16) by the retrieved treatment result data if aberrations have been generated by the treatment in these corneal treatments; adapting (S18) the initial correction parameters for treating the cornea (16) depending on the aberrations ascertained from the treatment result data; and providing (S20) the control data, which includes the adapted correction parameters.

### FIELD

The invention relates to a method for providing control data for an ophthalmological laser of a treatment apparatus for treating a cornea of a human and/or animal eye. Furthermore, the invention relates to a control device, which is configured to perform the method, to a treatment apparatus with such a control device, to a computer program comprising commands, which cause the treatment apparatus to execute the method, and to a computer-readable medium, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device may, for example, be formed such that laser pulses effect a photodisruption and/or ablation in a focus area situated within the organic tissue to remove a tissue, in particular a tissue lenticule, from the cornea.

In the treatment of the eye with the ophthalmological laser, it can occur that aberrations, in particular spherical aberrations, are newly induced by the corrections on the cornea, which have not yet been present before. Such newly induced aberrations present disturbing side effects, which can hardly be estimated before the treatment.

### SUMMARY

It is the object of the invention to provide an improved correction of the cornea, in particular while avoiding newly generated aberrations.

This object is solved by the examples provided herein. Advantageous embodiments of the invention are disclosed in the dependent claims, the following description as well as the figures.

The invention is based on the idea that newly introduced aberrations may be compensated for. From statistics of pretreatments which have identical treatment parameters and physiological conditions, it is ascertained which aberrations usually arise, wherein these aberrations may then be taken into account and compensated for in favor of the individual treatment.

An aspect of the invention relates to a method for providing control data for an ophthalmological laser of a treatment apparatus for treating a cornea of a human and/or animal eye, wherein the method comprises the following steps performed by a control device. Therein, an appliance and/or an appliance component, in particular a computer and/or processor, may perform the following steps in automated and/or semi-automated manner, that is with input of data and/or commands, is meant by a control device: determining eye parameters from predetermined examination data; determining initial correction parameters for treating the cornea depending on the eye parameters; determining treatment result data by a database, which includes treatment results of preceding corneal treatments, wherein the treatment result data is retrieved from treatments with comparable eye parameters and initial correction parameters; determining by the retrieved treatment result data if aberrations have been generated by the treatment in these corneal treatments; adapting the initial correction parameters for treating the cornea depending on the aberrations ascertained from the treatment result data; and providing the control data, which includes the adapted correction parameters.

In other words, eye parameters, which the eye and/or the cornea have before the treatment, may first be ascertained from predetermined examination data with the aid of the control device. As the examination data, topography measurements, wavefront measurements and/or subjective visual disorder data, which may in particular be ascertained with a phoropter, may, for example, be predetermined. From this examination data, the eye parameters may then be ascertained, which, for example, indicate curvatures in the form of sphere, cylinder and/or axis values, initial aberrations and/or subjective refraction values. In other words, the eye parameters may include values, which serve as a basis for the treatment with the ophthalmological laser.

Then, correction parameters for treating the cornea may be determined therefrom to, for example, correct a visual disorder ascertained from the eye parameters. In particular, an aspherical correction of the cornea may be planned. In particular, the correction parameters may include refraction values, in particular sphere, cylinder and axis correction values, an optical zone, a centering of the treatment and/or curvature values (K-Readings) to be achieved.

Subsequently, a database of patients who comprise the same or similar eye parameters, who have already been treated with the same or similar correction parameters, may be examined, wherein the treatment results have been predetermined for these patients and are stored in the database. If it is ascertained that aberrations have been generated by the treatment in one or more of these patients, in particular a spherical aberration, coma or trefoil, these aberrations may be previously compensated for by an adaptation of the correction parameters, such that a new aberration is not introduced by the treatment. Thereto, statistics may be created from the treatment result data present in the database, which aberrations have occurred in the respective case, wherein the aberrations statistically most frequently introduced may thus be previously compensated for. If it is determined that aberrations were not introduced in treatments, an adaptation of the initial correction parameters is not further required.

Initial correction parameters refers to those corrections, which were originally planned for treating the visual disorder, which may be ascertained from the eye parameters. Accordingly, the adapted correction parameters further include the correction of the visual disorder plus a compensation for the aberrations to be expected by the treatment.

The treatment result data, which may be predetermined in the database, may in particular be gathered based on medical aftercare examinations after the treatment and provide a statement about the treatment success and/or newly introduced aberrations.

By the invention, the advantage arises that aberrations to be expected may be previously compensated for in a simple manner, which improves the treatment with the ophthalmological laser of the treatment apparatus.

The invention also includes embodiments, by which additional advantages arise.

In an embodiment, the correction parameter values for an aspherical refraction correction in particular include a sphere value, a cylinder value, an axis value and/or an addition value, and/or parameters of a planned optical zone, in particular a diameter and/or a centering of the optical zone, and/or a planned curvature value. In other words, the correction parameters provide values, which are provided for the correction of a visual disorder and for planning the treatment.

In a further embodiment, the eye parameters include a corneal thickness and/or a preoperative curvature value of the cornea and/or a thickness of an epithelial layer and/or visual disorder values. Herein, visual disorder values refer to subjectively ascertained visual disorder values, which may, for example, be determined by a phoropter.

In a further embodiment, higher order aberrations, in particular a spherical aberration, coma and/or trefoil, are ascertained from the treatment result data and compensated for by adapting the correction parameters. This means that the treatment result data provided in the database may include newly introduced aberrations, in particular higher order aberrations, which have, for example, been determined by follow-up examinations.

In a further embodiment, a statistical value of the aberrations of all of the treatment results with comparable eye parameters and initial correction parameters is ascertained with the aid of the retrieved treatment result data, wherein the adaptation of the initial correction parameters is performed based on the statistical value. In other words, the treatment result data of all of the preceding corneal treatments may be statistically evaluated, in which the same or similar eye parameters and initial correction parameters are present or have been used. The statistical value may, for example, include an average value or a median value. For example, it may be known from the treatment result data that a spherical aberration is generated with present eye parameters and initial correction parameters, wherein the statistical value may indicate a degree of severity of the spherical aberration from all of the preceding treatments. Thus, the compensation for this aberration may be planned in an improved manner.

A method for controlling a treatment apparatus may be further provided. Therein, the method may include the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method may include at least one additional step, which is executed if and only if an application case or an application situation occurs, which has not been explicitly described here. For example, the step may include the output of an error message and/or the output of a request for inputting a user feedback. Additionally or alternatively, it may be provided that a default setting and/or a predetermined initial state are adjusted.

A further aspect of the invention relates to a control device, which is configured to perform the steps of at least one embodiment of one or both of the previously described methods. Thereto, the control device may comprise a computing unit for electronic data processing such as, for example, a processor. The computing unit may include at least one microcontroller and/or at least one microprocessor. The computing unit may be configured as an integrated circuit and/or microchip. Furthermore, the control device may include an (electronic) data memory or a storage unit. A program code may be stored on the data memory, by which the steps of the respective embodiment of the respective method are encoded. The program code may include the control data for the respective laser. The program code may be executed by the computing unit, whereby the control device is caused to execute the respective embodiment. The control device may be formed as a control chip or control unit. The control device may for example be encompassed by a computer or computer cluster.

A further aspect of the invention relates to a treatment apparatus with at least one eye surgical or ophthalmological laser and a control device, which is configured to perform the steps of at least one embodiment of one or both of the previously described methods. The respective laser may be configured to at least partially separate a predefined corneal volume with predefined interfaces, of a human or animal eye, by optical breakdown, in particular at least partially separate it by photodisruption and/or to ablate corneal layers by (photo) ablation and/or to effect a laser-induced refractive index change in the cornea and/or the eye lens and/or to increase a cross-linking of the cornea.

In a further embodiment of the treatment apparatus according to the invention, the laser may be suitable to emit laser pulses in a wavelength range between 300 nm and 1400 nm, for example between 900 nm and 1200 nm, at a respective pulse duration between 1 fs and 1 ns, for example between 10 fs and 10 ps, and a repetition frequency of greater than 10 kilohertz (kHz), for example, between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakdown may be spatially narrowly limited such that a high incision accuracy is allowed in the generation of the interfaces. In particular, the range between 700 nm and 780 nm may also be selected as the wavelength range.

In a further embodiment of the treatment apparatus according to the invention, the control device may comprise at least one storage device for at least temporary storage of at least one control dataset, wherein the control dataset or datasets include(s) control data for positioning and/or for focusing individual laser pulses in the cornea; and may comprise at least one beam device for beam guidance and/or beam shaping and/or beam deflection and/or beam focusing of a laser beam of the laser.

A further aspect of the invention relates to a computer program. The computer program includes commands, which for example form a program code. The program code may include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above-mentioned computer program and the commands thereof, respectively, are stored. For executing the computer program, a computer or a computer cluster may access the computer-readable medium and read out the content thereof. The storage medium is for example formed as a data memory, in particular at least partially as a volatile or a non-volatile data memory. A non-volatile data memory may be a flash memory and/or an SSD (solid state drive) and/or a hard disk. A volatile data memory may be a RAM (random access memory). For example, the commands may be present as a source code of a programming language and/or as assembler and/or as a binary code.

Further features and advantages of one of the described aspects of the invention may result from the embodiments of another one of the aspects of the invention. Thus, the features of the embodiments of the invention may be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following may be present in any combination with each other and/or the features of the embodiments. This means, the features of the execution examples may supplement and/or replace the features of the embodiments and vice versa. Thus, configurations are also to be regarded as encompassed and disclosed by the invention, which are not explicitly shown or explained in the figures, but arise from and may be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematic method diagram for providing control data according to an exemplary embodiment.

### DETAILED DESCRIPTION

In the figures, identical or functionally identical elements are provided with the same reference characters.

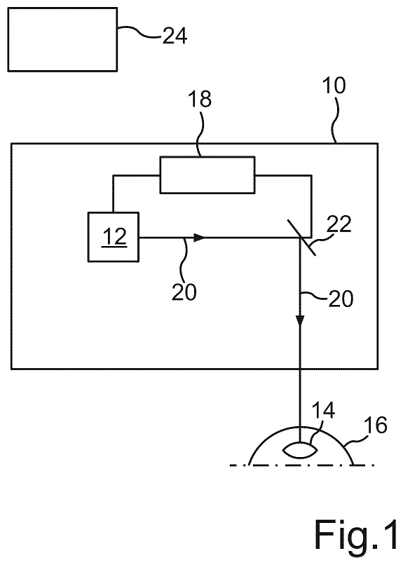

FIG. 1 shows a schematic representation of a treatment apparatus 10 with an ophthalmological laser 12 for removing a tissue or volume body 14 from a human or animal cornea 16 by photodisruption and/or ablation. The volume body 14 may for example represent a lenticule, which can be separated from the cornea 16 by the eye surgical laser 12 for correcting a visual disorder. Correction parameters or a geometry of the volume body 14 to be removed may be provided by a control device 18, in particular in the form of control data, such that the laser 12 emits pulsed laser pulses in a pattern predefined by the control data into the cornea 16 of the eye to remove the volume body 14. Alternatively, the control device 18 may be a control device 18 external with respect to the treatment apparatus 10.

Furthermore, FIG. 1 shows that the laser beam 20 generated by the laser 12 may be deflected towards the cornea 16 by a beam deflection device 22 such as for example a rotation scanner, to remove the volume body 14. The beam deflection device 22 may also be controlled by the control device 18 to remove the volume body 14.

In particular, the illustrated laser 12 may be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 femtoseconds and 10 picoseconds, and a repetition frequency of greater than 10 kilohertz, for example between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

Furthermore, a database 24 is illustrated, which may belong to the treatment apparatus 10 or may be provided externally thereto. In particular, the control device 18 may comprise a data link to the database to thus obtain data from it. In the database, treatment result data may be stored, which has treatment results of preceding corneal treatments. This means that treatment successes or complications, in particular the development of new aberrations, which have only been generated by the treatment, may be stored in the form of treatment result data.

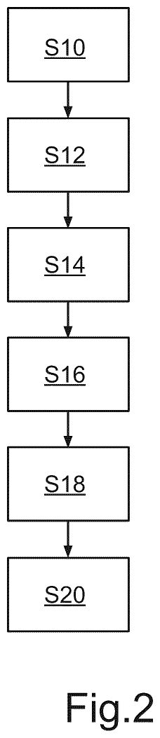

In laser treatments, it can possibly occur that after the volume body 14 has been removed from the cornea 16, aberrations are induced by the treatment with the treatment apparatus 10, which may impair the treatment result, for example, due to a decentration of the volume body 14 or other effects. In order to reduce or avoid the development of new aberrations in such a treatment, it may therefore be provided that the control device 18 is formed to perform the method shown in FIG. 2.

FIG. 2 shows a schematic method diagram for providing control data for an ophthalmological laser 12 of a treatment apparatus 10 for treating a cornea 16.

In a step S10, eye parameters may be ascertained from predetermined examination data. Herein, the examination data may be recorded before the treatment within the scope of diagnostic measurements, wherein the eye parameters, for example, include a corneal thickness and/or a preoprative curvature value of the cornea and/or a thickness of an epithelial layer and/or visual disorder values, in particular subjectively determined visual disorder values. For example, already present aberrations may also be determined from the examination data as the eye parameters. The examination data may originate from an external device, for example, from a database 24.

In a step S12, initial correction parameters for treating the cornea may be determined depending on the ascertained eye parameters. The initial correction parameters may indicate values which are required to compensate for the visual disorder of the eye ascertained from the eye parameters. For example, the values indicated may include an aspherical refraction correction, a diameter of a planned optical zone, or a centering of an optical zone and/or curvature values, which the cornea is to have after the treatment.

In a step S14, the control device 18 may in particular retrieve treatment result data from the database 24, in which treatment results of preceding corneal treatments may be stored, for example, of corneal treatments, in which comaprable eye parameters and initial correction parameters have been present before the treatment.

This treatment result data may then be examined in a step S16 to the effect if aberrations have arisen by the treatment in these comaprable treatments. In particular, it may be examined by statistics from the treatment result data, which aberrations and to what extent, this means the degree of the aberrations, have arisen in the respective treatment.

In a step S18, the initial correction parameters may then be adapted such that the aberrations to be expected, in particular according to their statistics, are compensated for by adaptation of the correction parameters and thus do not even arise in the treatment by the treatment apparatus 10.

Finally, control data for controlling the ophthalmological laser 12 may be provided in a step S20, which has been generated based on the adapted correction parameters. The treatment apparatus 10, in particular the ophthalmological laser 12 and/or the beam deflection device 22, may then be controlled by the control data for removing the volume body 14 by the adapted correction parameters.

Overall, the examples show how an aspherical compensation may be provided by the invention.

1. A method for providing control data for an ophthalmological laser of a treatment apparatus for treating a cornea of a human and/or animal eye, wherein the method comprises the following steps performed by a control device:
determining eye parameters from predetermined examination data;
determining initial correction parameters for treating the cornea depending on the eye parameters;
retrieving treatment result data from a database which includes predetermined treatment results of preceding corneal treatments, wherein the treatment result data is retrieved from the preceding corneal treatments with comparable eye parameters and initial correction parameters;
determining from the retrieved treatment result data if aberrations have been generated by the preceding corneal treatments;
adapting the initial correction parameters for treating the cornea depending on the aberrations ascertained from the treatment result data;
providing the control data, which includes the adapted correction parameters.

2. The method according to claim 1, wherein the initial correction parameters include values for an aspherical refraction correction, and/or parameters of a planned optical zone, and/or a planned curvature value.

3. The method according to claim 1, wherein the eye parameters comprise at least one of: a corneal thickness, a preoperative curvature value of the cornea, a thickness of an epithelial layer, and visual disorder values.

4. The method according to claim 1, wherein higher order aberrations coma and/or trefoil, are ascertained from the treatment result data and compensated for by the adaptation of the initial correction parameters.

5. The method according to claim 1, wherein a statistical value is ascertained of all of the aberrations generated in the preceding corneal treatments with comparable eye parameters and initial correction parameters, wherein the adaptation of the initial correction parameters is performed based on the statistical value.

6. A control device, which is configured to perform the method according to claim 1.

7. A treatment apparatus with at least one eye surgical laser for the separation of a corneal volume of a human or animal eye by optical breakdown, and at least one control device according to claim 6.

8. (canceled).

9. A computer-readable medium for storing a computer program, the computer program comprising commands which cause a treatment apparatus to execute a method according to claim 1.

10. The method according to claim 2, wherein the aspherical refraction correction includes a sphere value, a cylinder value, an axis value and/or an addition value.

11. The method according to claim 2, wherein the parameters of the planned optical zone include a diameter and/or a centering of the optical zone.

12. The method according to claim 4, wherein the higher order aberrations include a spherical aberration, a coma and/or a trefoil.
